(19) United States
(12) Patent Application Publication
(10) Pub. No.: US 2009/0318572 A1
(43) Pub. Date: Dec. 24, 2009

(54) APPARATUS AND PROCESS FOR PRODUCTION OF LIQUID FUEL FROM BIOMASS

(76) Inventors: Masayasu Sakai, Nagasaki (JP); Nobuaki Murakami, Nagasaki (JP); Toshiyuki Takegawa, Nagasaki (JP); Hachiro Kawashima, Nagasaki (JP)

Correspondence Address: MASAO YOSHIMURA, CHEN YOSHIMURA LLP, 333 W. El Camino Real, Suite 380, Sunnyvale, CA, 94087, US

(21) Appl. No.: 12/517,806

(22) PCT Filed: Dec. 5, 2007
(86) PCT No.: PCT/JP2007/073523

§ 371 Date: Jun. 4, 2009

(30) Foreign Application Priority Data
Dec. 5, 2006 (JP) 2006-328155

(51) Int. Cl. C07C 27/20; B01J 19/00

(52) U.S. Cl. 518/700; 422/189

(57) ABSTRACT

The liquid fuel synthesizing apparatus (1) for efficient synthesis of liquid fuel from biomass, such as plant for a raw material, comprised of reactors (2) with a catalyst incorporated therein, coolers (3) for cooling of synthesized gaseous matter and extracting of liquid fuel, and temperature adjustment means (7) for regulating by heat exchange any unreacted gas after recovery of liquid fuel to reaction temperature suitable for liquid fuel gas synthesis, wherein the reactors, the coolers and the temperature adjustment means are arranged multistage inline-straightly along gas flow. The temperature adjustment means (7) and the reactors (2) are disposed in the constant temperature room (8) in parallel. The coolers (3) are disposed apart from the constant temperature rooms (8).

### TECHNICAL FIELD

The present invention relates to an apparatus and a production process for synthesizing liquid fuel from biomass such as plants as a raw material.

### TECHNICAL BACKGROUND

When considering the shortage and depletion of oil resources and the global warming issue mainly due to CO₂, the importance of the selection and development of the substitutes cannot be overstressed. Since a long-term forecast shows that an enormous amount of substitutes is required, large expectations have been placed, from a quantitative viewpoint, on the production of synthetic fuel made from biomass as a raw material.

As in a case where natural gas and coal are used as raw material, liquid fuel (e.g., methanol, DME (dimethyl ether), or hydrocarbon by F-T (Fischer-Tropsch) process) can be obtained from synthetic gas (H₂+CO) obtained by using biomass as a raw material to gasify the material. These liquid fuels synthesized from biomass have advantages, including: (1) these fuels are obtained through the accumulation of solar power and can be subjected to cyclic regeneration, (2) biomass as a raw material is not unevenly distributed all over the world and thus can be expected to be available in the future in an amount equal to that of the current consumption of petroleum, (3) these fuels are storable and portable in a favorable manner, and (4) these fuels do not increase CO₂ in air and does not include a sulfur content and thus is clean for example.

However, as described by the present inventor in Non-patent Document 1, in spite of the fact that a great number of researches and developments have been made, the current situation where the production of synthetic fuel using biomass as a raw material has not been realized at an industrial scale is due to the following reasons. First, biomass has not been highly recognized until recently. Secondly, since biomass includes oxygen, the calorific value is low and thus such synthetic gas required for the production of liquid fuel could not be obtained that had a high calorific value and that included H₂ at a high content ratio. Non-patent Document 1 is a document by the present inventors describing the prior art (regarding large-scale apparatus).

A process for producing liquid fuel from synthetic gas obtained by gasifying biomass is basically the same process for producing liquid fuel from fossil fuel such as natural gas or coal as raw material. Specifically, a production process using natural gas as a raw material is mainly composed of a step of converting fossil fuel as raw material to synthetic gas of carbon monoxide and hydrogen for example to subsequently remove impurities such as sulfur and unwanted matters such as CO₂ in synthetic gas, a step of causing the resultant synthetic gas to react under the existence of a catalyst to convert the gas to liquid fuel, and a step of refining liquid fuel to have a property of a target fuel to obtain a product.

Gasification methanol synthesis, which is one of processes for producing liquid fuel from biomass, has been subjected to many experiments and has reached an industrial scale up to the step of gasifying biomass. However, the gasification methanol synthesis has not reached a level at which methanol is synthesized efficiently. The reason is that biomass has a low calorific value as described above, thus failing to provide synthetic gas having a high calorific value required for the methanol synthesis. However, advances have been seen even in the biomass gasification technique and thus some promising aspects have been found in the production of synthetic gas (e.g., Patent Document 1).

Another point to which attention should be paid for the production of synthetic gas is a capacity. For example, a current methanol synthesis apparatus using natural gas as a raw material has a production scale of 1,000 to 2,000 tons/day and is operated, from the viewpoints of energy efficiency and economic efficiency, at a high pressure of 5 MPa or more and frequently at 6 MPa or more. However, a biomass integration density in Japan in particular is not so high. Thus, a methanol production process and a production apparatus are required that can cope with a production scale of 0.1 to 100 tons/day one digit to four digits smaller than the above methanol production scale, that are small in size, and that can be operated easily at a low pressure.

As a methanol production technique that is small and that can be operated easily at a low pressure, the technique of Patent Document 2 has been already known. This technique is suggested by the present inventors and discloses a methanol synthesis apparatus in which methanol synthetic reactors are serially coupled at a plurality of stages. Although this invention discloses that the same operation is repeated at many stages, the existence of the reactors at a plurality of stages has prevented these reactors from having an appropriate temperature adjusted for reaction. Furthermore, due to the longitudinal layout in which the reactors are arranged in the longitudinal direction from the top to bottom, an exchange of catalysts has required the disassembly of reactors in all stages and thus has required a long-time maintenance and thus an inconvenience also has been caused in the operation schedule. Furthermore, due to the layout in which temperature controlling devices are parallely arranged in all stages, a disadvantage has been caused that the resultant apparatus is complicated.

Furthermore, when liquid fuel gas is synthesized from synthetic gas obtained by gasifying biomass (e.g., when methanol gas is synthesized), higher concentrations of H₂ and CO in the synthetic gas provide a higher yield of methanol gas. If these concentrations are low, methanol gas cannot be synthesized at a high yield. Thus, in order to efficiently produce methanol from biomass, what is required is, in addition to a methanol synthesis apparatus, a biomass gasification technique appropriate for the apparatus and a technique of producing synthetic gas including H₂ and CO or CO₂ at a high concentration in particular.

Furthermore, when considering the use of biomass as an energy plantation, in order to increase the amount of the production of methanol per a unit area, it is advantageous to use, from among the synthetic gas obtained by the gasification of biomass, not only carbon monoxide but also carbon dioxide as a raw material for synthesizing liquid fuel. In recent years, a catalyst by which methanol can be synthesized from carbon dioxide and hydrogen has been developed. This catalyst has a higher performance than that of a catalyst for methanol synthesis from carbon monoxide and hydrogen. Furthermore, only synthetic gas obtained from biomass cannot provide a sufficient amount of hydrogen to synthesize methanol from carbon dioxide. It has been clarified that, as a system that can supplement this shortage, a system is effective that uses electrolytic hydrogen using hydraulic power generation, wind-power generation, and photovoltaic power generation for example.

### Patent Document 1: Japanese Unexamined Patent Application Publication No. 2004-051718

### Patent Document 2: Japanese Unexamined Patent Application Publication No. 2005-132739

Non-patent Document 1: Kusaki Baiomasu Karano Gousei Gasu Seizou To Ekitai Nenryou Gousei (Journal of The Japan Institute of Energy, volume 81, No. 12, p. 1063-1068)

### DISCLOSURE OF THE INVENTION

### Problem to be Solved by the Invention

In view of the above situation, it is an objective of the present invention to provide a process for producing liquid fuel from synthetic gas obtained by gasifying biomass such as plants as a raw material and a liquid fuel synthesis apparatus that is small in size and operates in a favorable manner and that achieves a high liquid fuel yield even at a low pressure operation. It is another objective of the present invention to provide a liquid fuel production apparatus including a biomass gasification apparatus highly compatible with a liquid fuel synthesis apparatus.

### Means for Solving Problem

The liquid fuel production process of the present invention is a process for producing liquid fuel from synthetic gas including hydrogen and carbon monoxide obtained by gasification of biomass. This process includes: a first step of causing the synthetic gas to have a contact with catalyst while being pressurized at a pressure of 0.5 to 5 MPa to synthesize liquid fuel gas; a second step of liquefying the liquid fuel gas to collect the gas to separate the gas from unreacted synthetic gas; a third step of again causing the unreacted synthetic gas to have a contact with catalyst while being pressurized at a pressure of 0.5 to 5 MPa to synthesize liquid fuel gas; and a step of repeating the first step and the third step. Liquid fuel includes liquid hydrocarbon fuel such as methane, ethane, or propane and alcohol fuel such as methanol.

The apparatus for synthesizing liquid fuel from biomass according to the present invention includes: a plurality of reactors and a plurality of coolers that are arranged in a serial manner, the reactors and the coolers are connected to one another via a synthetic gas supply pipe and a reaction gas derivation pipe, and the coolers include liquid fuel collection pipes.

The apparatus for producing liquid fuel from biomass according to the present invention includes: a biomass supply hopper; a gasification reaction apparatus; and a liquid fuel synthesis apparatus. The gasification reaction apparatus includes therein a secondary gasification reaction pipe and a primary gasification reaction room that has a gasification agent supply line linked to the secondary gasification reaction pipe. The liquid fuel synthesis apparatus is composed of a plurality of reactors and a plurality of coolers that are arranged in a serial manner, the reactors and the coolers are connected to one another via a synthetic gas supply pipe and a reaction gas derivation pipe, and the coolers include liquid fuel collection pipes. The biomass supply hopper is connected to the gasification reaction apparatus by being connected to the primary gasification reaction room via a biomass supply line. The gasification reaction apparatus is connected to the liquid fuel synthesis apparatus via a synthesis raw material gas supply line.

According to the process for producing liquid fuel of the present invention, the synthesis of liquid fuel gas by synthetic gas and a liquid fuel synthesis catalyst, the liquefaction of synthesized liquid fuel gas, and a process for separating unreacted synthetic gas are performed repeatedly. Thus, the synthesized liquid fuel gas is removed from the reaction system at every synthesis.

When the liquid fuel gas is hydrocarbon gas, the equilibrium relation of Fischer-Tropsch reaction shown by the following formula is promoted in the arrow direction. By repeating this a plurality of times, the synthesis yield of hydrocarbon gas can be increased.

(2n+1)H₂+nCO→CₙH₂ₙ₊₂+H₂O

Fischer-Tropsch reaction generally uses a catalyst of a compound of iron or cobalt. For example, a cobalt-supporting catalyst can be obtained by impregnating cobalt nitrate in silica gel to subsequently dry the silica gel to burn the silica gel at 400 degrees C. for 2 hours.

When the liquid fuel gas is methanol gas, this means that the equilibrium relation of the methanol synthesis reaction shown by the following formula is promoted in the arrow direction. By repeating this a plurality of times, the synthesis yield of methanol can be increased.

2H₂+CO→CH₃OH

Currently, methanol synthesis is operated by mainly copper or zinc-base catalyst and at 200 to 260 degrees C. and at a high pressure of 5 MPa or more and frequently 6 MPa or more. The reason why the synthesis is performed under a high pressure condition is that, due to the equilibrium relation of the methanol synthesis reaction using a catalyst, a low pressure condition causes a low methanol yield (i.e., a low rate at which hydrogen and carbon monoxide are converted to methanol). However, such an operation under a high pressure is desirably avoided because, setting aside a large-size apparatus, a small-size synthesis apparatus subjected to an operation at a high pressure results in a high load on the apparatus and the safety may be insecure.

The liquid fuel synthesis apparatus of the present invention includes: a plurality of reactors filled with catalysts and a plurality of coolers that are arranged in a serial manner. The reactors and the coolers are connected to one another via a synthetic gas supply pipe and a reaction gas derivation pipe. The coolers include liquid fuel collection pipes. By the liquid fuel synthesis apparatus of the present invention, the synthesis yield of liquid fuel can be increased even under a low pressure.

A catalyst included in the reactor is not particularly limited and may be, for example, a catalyst used for methanol synthesis (e.g., zinc oxide, Cu/ZnO, Al₂O₃, ZrO₂, copper, zinc, aluminum, germanium, or manganese-base catalyst).

The synthetic gas separated in the final reactor still includes hydrogen, carbon monoxide, and hydrocarbon. These substances may be circulated to be used as synthetic gas or also may be used as gas fuel.

Any cooler may be used so long as the cooler has a function to cool the gaseous liquid fuel obtained through the reaction to a temperature equal to or lower than the liquefaction temperature. The cooler may be the one for subjecting gaseous cooling medium or liquid cooling medium to a heat exchange or also may be stored in a cooling bath filled with cooling water of a predetermined temperature or lower. Gaseous methanol is cooled by the cooler to have a temperature equal to or lower than the liquefaction temperature and is liquefied. The liquefied liquid fuel is removed from the reaction system by the liquid fuel collection pipe and is collected.

The liquid fuel synthesis apparatus of the present invention has a temperature adjustment means for adjusting the temperature of the reactor to an appropriate temperature.

The reactors also may be parallely arranged in a constant temperature room in which the temperature can be adjusted.

As described above, the synthesized gaseous liquid fuel is liquefied by being cooled and is separated from unreacted synthetic gas and is collected. Unreacted hydrogen and carbon monoxide in the separated synthetic gas is sent to the next reactor and is further synthesized into liquid fuel gas. However, even when the unreacted synthetic gas cooled in the cooler is sent to the next reactor, this unreacted synthetic gas includes local low-temperature parts in which an appropriate synthetic reaction is prevented from being promoted. Thus, it is important to heat the synthetic gas at the inlet of the reactor to have an appropriate temperature. Specifically, although not limited to a particular configuration, it is preferable that the previous stage of the reactor has a heat exchanger or the reactor is stored in a constant temperature room in which the temperature can be adjusted to thereby perform a stable adjustment of the temperature.

The reactor also may be the one that maintains the temperature in the reactor at an appropriate temperature by subjecting the reaction heat generated by the synthetic reaction to a heat exchange with the surrounding atmosphere.

The temperature in the constant temperature room can be controlled by a gaseous medium or air, or also by water vapor generated by a low-pressure boiler, for example. Alternatively, the heat dissipation capability also may be improved by circulating water vapor of 10 atmospheric pressure or less by a circulator to improve the circulation flow rate. When water vapor of 10 atmospheric pressure or less is used, vapor having absorbed heat discharged from the constant temperature room may be cooled by an air cooler for example at the outside of the constant temperature room to merge the vapor with new pressurized water vapor to circulate the resultant vapor.

In the case of the current large-size apparatus, the methanol synthesis reaction causes significant heat generation (21.7 kcal by generation of 1 mol of methanol). Thus, it is required to remove the generated heat to maintain a fixed reaction temperature. Since this heat removal method has an influence on the entire energy efficiency, various methods have been tried. A typical heat removal method currently performed in the industrial field is called a quench method according to which a plurality of catalysts are provided at a plurality of stages among which cool raw material gas is introduced. This method is simple but causes a high gas flow rate to thereby increase the resistance loss in the flow and also requires a large amount of catalyst. In the case of a small-size apparatus to which the present invention can be applied on the other hand, the situation is different from that of a large-size apparatus. Specifically, the rate of heat dissipation from the surface of the reactor increases in proportion to the heat generation by the reaction. Thus, the temperature of the catalyst section can be maintained at a fixed temperature by the heat dissipation from the outer face of the container of the reactor. This method is very advantageous in the gas flow rate and the catalyst amount.

However, there may be a case where not cooling but heating is required at the outer side of the reaction or a case where a small amount of cooling is required when a dissipation rate is insufficient compared to heat generation. This also may be influenced by the outside temperature or air flow for example. According to the present invention, in order to cope with any case, the constant temperature room stores therein a reactor to thereby maintain appropriate operation conditions, thus realizing a stable and reliable apparatus. Alternatively, fins for example also may be provided at the surface of the reactor to thereby efficiently perform a heat exchange.

The liquid fuel synthesis apparatus of the present invention is structured so that the reactor has an outer diameter of 20 cm or less for example. The liquid fuel synthesis apparatus is structured to include: a plurality of reactors for subjecting reaction heat generated by synthetic reaction to a heat exchange with the surrounding atmosphere to maintain an appropriate inner temperature and a plurality of temperature adjustment means for maintaining the gas at the inlet of the reactors at an appropriate temperature. The reactors and temperature adjustment means are parallely arranged in the constant temperature room.

The reason why the reactor has a diameter of 20 cm or less and preferably 15 cm or less is that a reactor having a large diameter makes it difficult to control the temperature of the interior and thus the center may be excessively heated due to reaction heat. On the other hand, a reactor having an excessively-small diameter causes a great number of reactors or an apparatus having high reactors, which is disadvantageous in cost. The cross-sectional area of the interior of the reactor is proportional to the square of the pipe diameter but the surface area of the outer wall of the pipe is merely proportional to the pipe diameter. Thus, a smaller pipe diameter is more advantageous for heat dissipation but causes a difficulty to fill catalyst in the pipe. To prevent this, in order to improve the heat dissipation capability per a catalyst volume in the reactor, no-catalyst spaces in which catalyst is not filled also may be provided with appropriate interval and length in the axial direction of the reactors.

Alternatively, a shell tube heat exchanger-type boiler also may be used that performs the cooling of the reactors or the constant temperature room by boiler water. In this case, the tube-side has the reactors are placed at tube side and the shell has the flowing boiler water. The boiler water itself is subjected to a liquid level control and a pressure control for maintaining a fixed pressure by a pressure control valve provided in a line sending discharged vapor. As a result, the shell-side temperature is maintained at a temperature at which vapor saturates. Specifically, the reactor-side temperature of about 250 degrees C. and the boiler-side temperature of about 230 degrees C. can provide the in-shell pressure of about 28 atmospheric pressure.

Instead of parallely arranging reactors at multiple stages, another layout is also possible where reactors are superposed at stages in the longitudinal direction. However, this longitudinal layout requires the reactors in all stages to be disassembled, which is disadvantageous in the maintenance and operation schedule, when a deteriorated catalyst needs to be exchanged with the new one. In addition, this longitudinal layout is also disadvantageous in that the apparatus is complicated and the assembling operation is difficult.

In contrast with this, according to the configuration of the present invention, the heating means and reactors at the respective stages are parallely arranged on a single floor face. Thus, when a certain catalyst is deteriorated, the deteriorated catalyst can be exchanged with the new one by opening only the reactor having the deteriorated catalyst through the upper section, which is significantly advantageous in the maintenance. The apparatus having this configuration also can be assembled easily.

The liquid fuel production apparatus of the present invention is structured so that a liquid fuel synthesis apparatus is linked to a biomass supply hopper and a gasification reaction apparatus for producing synthetic gas.

The gasification reaction apparatus has the following characteristics. A gasification space is provided in which the ground biomass supplied from the biomass supply hopper receives water vapor and is subjected to a gasification reaction. Without supplying oxygen to the gasification space, the gasification space is blocked by a partition wall from the outer heating space. The ground biomass is heated mainly by heat transfer by radiation. By using the heat for reaction heat, a gasification reaction between water vapor and biomass is caused to occur.

The gasification reaction apparatus includes therein a secondary gasification reaction pipe and a primary gasification reaction room that has a gasification agent supply line linked to the secondary gasification reaction pipe. The primary gasification reaction room is linked to the biomass supply hopper via the biomass supply line and is also linked to the liquid fuel synthesis apparatus via the synthetic gas supply pipe.

The synthetic gas obtained by the gasification reaction apparatus includes a great amount of hydrogen gas and carbon monoxide gas required for the synthesis of liquid fuel and can be obtained in a clean state free from tar. Thus, the liquid fuel synthesis yield is high and the apparatus can be stably operated without having a mechanical problem.

Conventionally, gasification of biomass has been performed by cutting wood to have a chip-like shape to supply the wood chips to a fixed bed or fluid bed-type gasification furnace to gasify the chips by a gasification agent of (O₂+H₂O). When this phenomenon is considered from the viewpoint of combustion, this phenomenon means that oxygen and wood are combusted to generate hot gas to use this hot gas to thereby subject the remaining wood to pyrolysis. This method provides a small amount of effective hydrogen and carbon monoxide and causes the generation of a great amount of trouble-causing polymeric tar. In order to decompose this tar, oxygen must be blown into tar to thereby subject tar to the secondary combustion. In this case, effective hydrogen and carbon monoxide cannot be allowed to remain. Specifically, when biomass having only a calorific value ½ to ⅓ smaller than that of a fossil fuel is used as a raw material, the synthesis of liquid fuel is very difficult.

The present inventors have made a success in using a new gasification method to minutely grind biomass raw material to mix the resultant powders with a gasification agent of (O₂+H₂O) in such a manner that the oxygen concentration of the gasification agent is reduced to an extremely-low value and is finally reduced to the oxygen concentration of zero to thereby achieve a jet flow floor (where microparticles are floating), thereby providing high-calorie gas. Furthermore, a bench scale experiment apparatus using this method has demonstrated that transparent and colorless clean gas including a great amount of effective hydrogen, carbon monoxide, and methane could be obtained without causing any tar or soot. This experiment also has demonstrated that liquid fuel could be synthesized from these gases.

The raw material was powders obtained by drying sorghum to subsequently grind sorghum and minute-and-dry spirulina powders. The former and latter raw materials showed methanol yields as a weight ratio to the raw materials of 49% and 60% that were calculated based on the gas composition. Thus, a yield of 50% can be expected even when an actual plant is assumed and the plant power is deducted. Methanol has a higher calorific value than that of biomass. Thus, when the methanol yield is evaluated based on the calorific value, the yield of 50% as a weight ratio corresponds to a yield of 60% calorific value.

According to this new gasification method, raw material is finely ground. Thus, not only large trees but also plants can be used as a raw material. This method does not need to consider sugar or starch as in a fermentation process.

The resultant gas has a composition significantly changing depending on the molar ratio [O₂]/[C] between carbon in the biomass and oxygen in the gasification agent. When complete combustion of [O₂]/[C]=1.0 is reached, the composition is naturally only CO₂. As [O₂]/[C] is smaller, the compositions of H₂ and CO increase and the methanol yield also increases. When [O₂]/[C]=0 is established, the methanol yield is maximum. In this case however, all the heat required to maintain the temperature field of the gasification furnace is provided from the outer side.

The methanol production apparatus of the present invention is structured so as to improve the methanol yield per biomass raw material, hydrogen obtained by water electrolysis by the power by renewable energy other than biomass is compensated to raw material gas for methanol synthesis.

According to the above configuration, departing from the basic objective of the present invention of the use of renewable energy, the compensated hydrogen can be used to convert carbon monoxide and carbon dioxide in synthetic gas to methanol to a maximum extent.

According to the methanol production apparatus of the present invention, in order to improve the methanol yield per biomass raw material, hydrogen obtained by water electrolysis, which power is obtained by renewable energy other than biomass, is compensated to the raw material gas for methanol synthesis. The synthetic gas obtained from the biomass includes hydrogen and carbon monoxide as well as carbon dioxide.

By the above configuration, carbon dioxide that may be raw material for methanol synthesis can be further increased by a certain amount, thus further increasing the methanol yield per biomass. In this case, however, the methanol reactor must use such a catalyst by which carbon dioxide can be used as a raw material for the synthetic reaction as described later.

### EFFECT OF THE INVENTION

According to the present invention, by serially arranging a plurality of reactors, high yield of synthesis liquid fuel gas is obtained since the temperature of methanol synthesis is maintained continuously in spite of a low pressure operation, and also the cooling system is simplified so as to prevent getting complex in spite of the existence of the plurality of reactors. Therefore, an improved apparatus is provided.

By the biomass gasification reaction apparatus of the present invention, synthetic gas suitable for the synthesis of liquid fuel gas can be obtained. This provides an effect according to which the liquid fuel yield is increased to improve the stability and reliability in the operation of the entire apparatus. Furthermore, synthetic gas contains higher content of carbon monoxide and carbon dioxide by adding hydrogen gas obtained by water electrolysis.

### BRIEF DESCRIPTION OF THE DRAWINGS

FIG. 1 illustrates a fuel gas synthesis apparatus according to a multistage basic experiment according to the first embodiment of the present invention.

FIG. 2 illustrates a graph showing the methanol conversion rate of the multistage basic experiment apparatus according to the first embodiment of the present invention.

FIG. 3 illustrates an example of a fuel gas synthesis apparatus according to the second embodiment of the present invention.

FIG. 4 schematically illustrates a process flow according to the second embodiment of the present invention.

FIG. 5 illustrates the configuration of the entire gasification reaction apparatus according to the third embodiment of the present invention.

FIG. 6 is a cross-sectional view illustrating a high-temperature hot gas generation apparatus that is a constituent element of a gasification reaction apparatus according to the third embodiment of the present invention.

FIG. 7 is a cross-sectional view illustrating the configuration of a biomass gasification reaction apparatus that is a constituent element of the gasification reaction apparatus according to the third embodiment of the present invention.

FIG. 8 illustrates a graph showing a synthetic gas composition ratio at a gasification reaction temperature of 900 degrees C. of the gasification reaction apparatus according to the third embodiment of the present invention.

FIG. 9 illustrates the configuration of the entire gasification reaction apparatus according to the fourth embodiment of the present invention.

### DESCRIPTION OF THE REFERENCE NUMERALS

1 Liquid fuel synthesis apparatus
2 Reactor
3 Cooler
4 Synthetic gas supply pipe
5 Reaction gas derivation pipe
6 Liquid fuel collection pipe
7 Temperature adjustment means
8 Constant temperature room
101 High-temperature hot gas generation apparatus
102 Combustion furnace
103 Fire grate
106 Primary air
107 Secondary air
108 Tertiary air
109 High-temperature combustion gas
201 Gasification reaction apparatus
202 Primary gasification reaction room
203 Secondary gasification reaction pipe
205 Biomass supply hopper
206 Coarse powder-accompanying gas supply line
203 Gasification agent supply line
A Synthesis raw material gas supply line
B High-temperature combustion gas supply line

### THE BEST MODE FOR CARRYING OUT THE CLAIMED INVENTION

The following section will specifically describe the present invention with reference to the drawings. In embodiments, a case where liquid fuel is methanol will be described as an example.

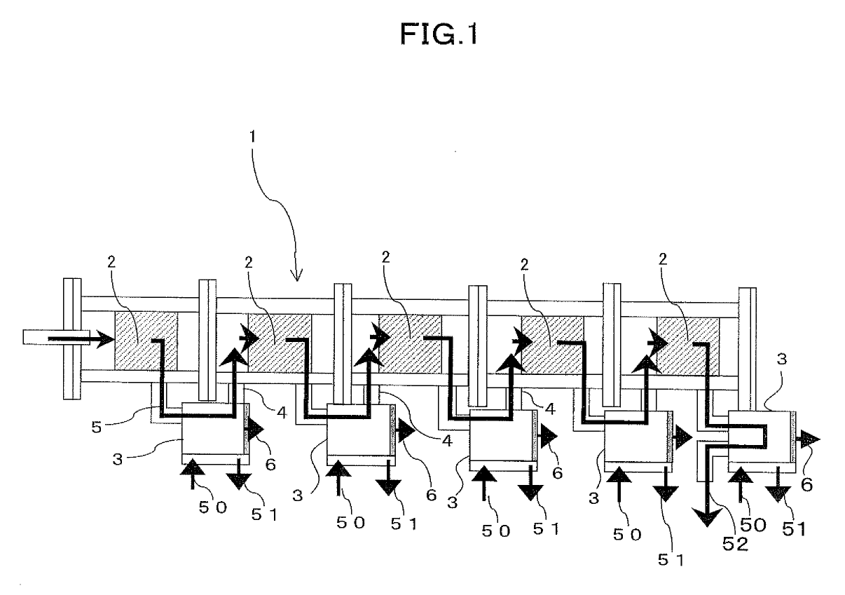

FIG. 1 schematically illustrates a multistage liquid fuel synthesis basic experiment apparatus that is the first embodiment according to the present invention. The liquid fuel synthesis apparatus is structured to include a plurality of reactors 2 generally arranged in 3 stages to 10 stages. In the shown example, the liquid fuel synthesis apparatus is composed of the reactors 2 in 5 stages. These reactors 2 are arranged serially with regard to the flow of gas.

The upstream-side of the reactor 2 is connected to a synthetic gas supply pipe 4 and the downstream-side is connected to a reaction gas derivation pipe 5 to be linked to a cooler 3. Synthetic gas, which is synthesis raw material of liquid fuel, is supplied from the synthetic gas supply pipe 4 to the reactor 2. The reactor 2 at the most upstream-side is linked to a synthesis raw material gas supply line A and receives the supply of synthetic gas. In the case of methanol synthesis, the reactor 2 for synthesizing liquid fuel is generally filled with copper or zinc-base methanol synthesis catalyst. However, in order to convert carbon dioxide to methanol, the reactor 2 is preferably filled with copper, zinc, aluminum, germanium, or manganese-base catalyst. Although synthetic reaction is achieved by exothermic reaction, heat dissipation from the surface of the container of the reactor 2 enables to maintain the reaction temperature in a range from 150 to 300 degrees C. The reaction gas includes methanol in the form of vapor. By using the cooler 3 to cool the reaction gas sent from the reaction gas derivation pipe 5 to the cooler 3, the methanol vapor included in the reaction gas is turned into liquid methanol to thereby remove liquid methanol as methanol through a liquid fuel collection pipe 6. The reference numeral 50 denotes a cooling water supply line and the reference numeral 51 denotes a discharge line.

The resultant synthetic gas from which this methanol vapor is removed and which can be further reactive under a chemical equilibrium is introduced to the reactor 2 of the next stage in which the same operation as that performed in the reactor 2 of the previous stage is performed. Then, the same step is repeated in multiple stages. The supplied synthetic gas is finally discharged through an unreacted gas discharge line 52 to the outside of the reactors.

Next, Experiment 1 showing the usefulness of the present invention was performed. Gasification was performed by a stainless steel-made gasification reaction pipe having an inner diameter of 50 mm and a length of 900 mm. Cedar wood powders obtained by grinding ceder to have a particle diameter of about 1 mm were supplied through the upper part of the reaction pipe at a speed of 2 g/minute and gasification agent of water vapor was supplied at 8 g/minute. The gasification reaction temperature was maintained at 1,000 degrees C. by electrically-heating the outer side of the reaction pipe. The cleanup of synthetic gas was performed by bubbling 0.1N sodium hydroxide solution. This gasification operation was continuously performed for one hour and synthetic gas was stored in a gas tank at an ordinary temperature. The gas in the tank had the following composition (volume %) under a dry gas standard.

H₂ 51.3, CO 35.7, CO₂ 7.3, CH₄ 3.1, C₂+2.6

In the above composition, C₂+ represents a component in which one molecule includes two or more carbons such as ethylene or ethane.

This gas was used as a raw material for methanol synthesis and was introduced to a methanol synthesis test apparatus according to the first embodiment. The reaction pipe is a stainless steel-made pressure-resistant reaction pipe that has an inner diameter of 67 mm and a height of 220 mm and is arranged in five stages. Each reaction pipe included a metal container to which 350 cc of copper-base catalyst and 350 cc of zinc-base catalyst were filled, respectively. The supply gas flow rate was 2Nl/minute. The gas flow is the same downward flow as that of FIG. 1 and the outlet of each stage has a reservoir in which gas after reaction is cooled and methanol is collected as liquid. The reaction pressure was adjusted to 0.9 MPa and the reaction temperatures in all of the five stages were adjusted to 200 to 220 degrees C. by a heating heater wound around the outer side of the reaction pipe. The reaction temperature is generally set in a range from 150 degrees C. to 300 degrees C.

The test was performed for 60 minutes. As a result, methanol in crude methanol could be obtained in an amount of 17.2 g, in spite of such a low pressure, a high yield of 40% (based on an amount of hydrogen supplied to the methanol synthesis apparatus) could be obtained.

Next, as a comparison example, the supply of cooling water was stopped and, without performing the liquefication and collection of methanol vapor in each stage, the experiment was performed so that only the gas from the outlet at the final stage was cooled. The result of the comparison example shows that the methanol yield was only 4.8 g per 60 minutes, thus showing the usefulness of the present invention.

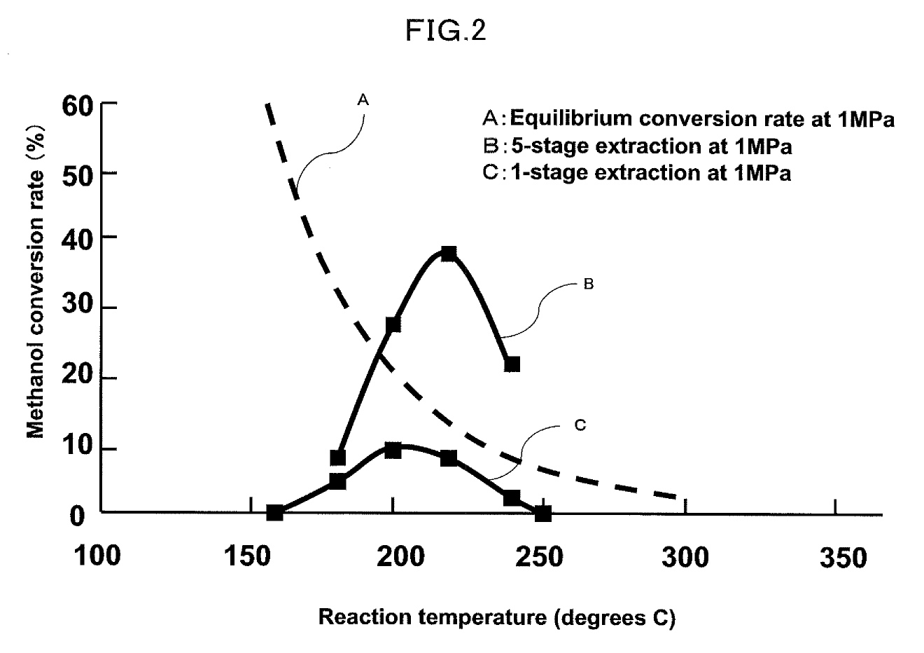

The above experiment result can be summarized by a methanol conversion rate as shown in FIG. 2. FIG. 2 shows a comparison between the performance B regarding a case where methanol is synthesized through multiple stages and the performance C regarding a case where one-stage extraction is used without cooling water flowing in the cooler that does not have a cooling function. While the one-stage extraction C shows a performance at the lower side of the equilibrium conversion rate curve A, the multiple-stage extraction B shows a performance far above from this curve A. Specifically, this shows that, a low pressure operation suitable for a small-size apparatus can provide a greater amount of methanol by the adjustment of the chemical equilibrium relation.

Next, Experiment 2 was performed in order to confirm the usefulness of an addition of a water electrolysis hydrogen production apparatus by natural energy such as water power or wind power. In Experiment 2, hydrogen gas from water electrolysis was simulated by additionally supplying the hydrogen gas from a purchased cylinder to the supply gas of Experiment 1 at a flow rate of 0.4 Nl/minute to thereby adjust the H₂/CO molar ratio to 2.0 optimal for the methanol synthesis. The other structures in Experiment 2 are the same as those of Experiment 1. As a result, 24.1 g of methanol could be obtained as expected after 60 minutes, i.e., methanol about 40% larger than that in Experiment Example 1 could be obtained. The methanol synthesis catalyst in this case was copper-base, zinc-base, aluminum-base, germanium-base, or manganese-base catalyst.

Here, a difference in the methanol yield will be shown between a case where only synthetic gas is used and a case where hydrogen is mixed in synthetic gas for compensation. The composition vol % of synthetic gas is calculated as an example.

A biomass informal molecular formula is assumed as C1.3H2O0.9 and a methanol conversion rate is assumed as 80%.

### Synthetic Gas Composition

H₂: 50%, CO: 20%, CH₄: 10%, C₂H₄: 1%, CO₂: 14%, N₂: 5%

Generally, CO₂ is not used as a main raw material for methanol synthesis but may be used as a raw material depending on a catalyst (e.g., copper-base, zinc-base, aluminum-base, germanium-base, or manganese-base catalyst). This is represented by reaction formulae as shown below.

CO+2H₂→CH₃OH

CO₂+3H₂→CH₃OH+H₂O

Methanol weight yields R in the respective cases can be calculated by the calculations as shown below.

When hydrogen is not compensated:

R=0.8×(CO)×1.3(C)/[(CO)+(CH₄)+2(C₂H₄)+(CO₂)]=0.8×20×1.3/[(20+10+2+14)]=0.452

Specifically, this means that 0.45 kg of methanol can be obtained from 1 kg of biomass. When hydrogen is compensated:

R=0.8×1.3(C)[CO+CO₂]/[(CO)+(CH₄)+2(C₂H₄)+(CO₂)]=0.8×1.3×[20+14]/[(20+10+2+14)]=0.768

Specifically, this means that 0.77 kg of methanol can be obtained from 1 kg of biomass.

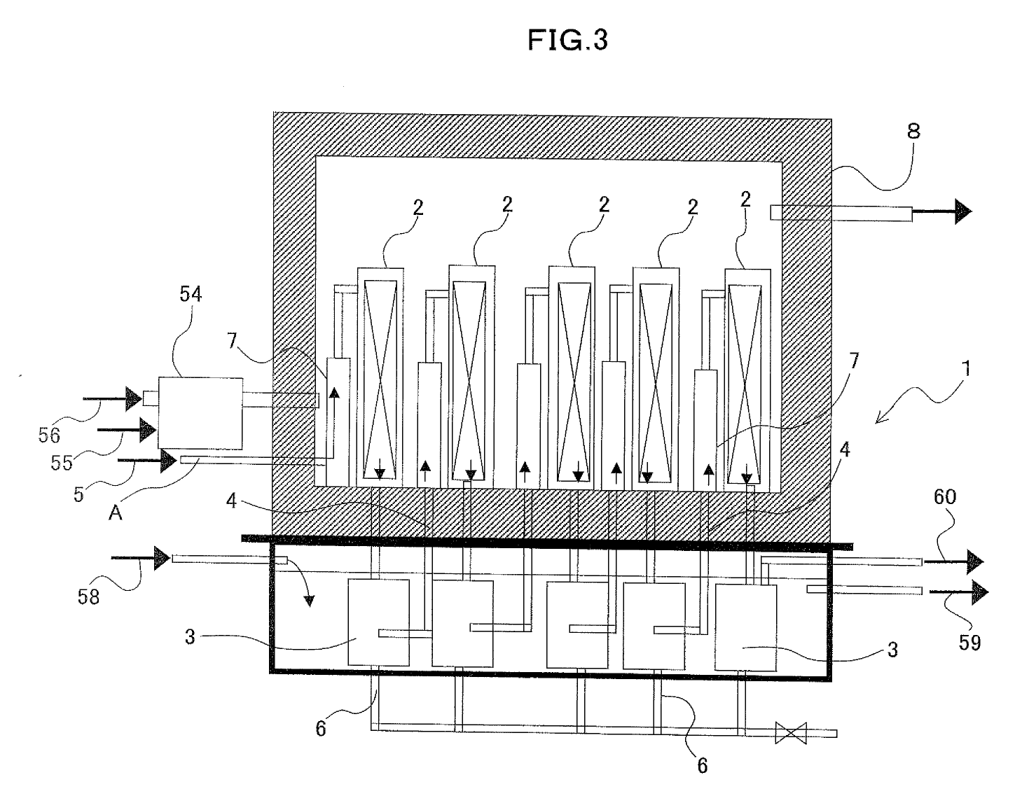

Next, the second embodiment of the liquid fuel synthesis apparatus having the main function of the present invention is shown in FIG. 3. The liquid fuel synthesis apparatus is composed of: the reactors 2 including therein catalysts and being made of stainless steel; the coolers 3 for cooling synthesized liquid fuel gas to extract liquid methanol; and temperature adjustment means 7 for adjusting, by heat exchange, the synthetic gas including an unreacted gas component left after the collection of liquid methanol to have a reaction temperature suitable for the synthesis. These members are arranged in a serial manner along the gas flow direction at multiple stages. The temperature adjustment means 7 and the reactors 2 are stored in a constant temperature room 8 having a heat insulating structure surrounded by a heat insulating material.

The coolers 3 on the other hand are stored in a cooling bath 53 positioned at the lower side of the constant temperature room 8.

Although this example shows a layout in which the constant temperature room and the cooling bath are provided at upper and lower sides respectively, the constant temperature room and the cooling bath also may be arranged to be adjacent to each other if the planar arrangement is convenient.

The constant temperature room 8 is set to have an appropriate temperature of 150 to 300 degrees C. by a constant temperature room temperature adjuster 54 using air and combustion gas. The reference numeral 55 denotes an air supply line. The reference numeral 56 denotes a combustion gas supply line. The reference numeral 57 denotes a discharge line for air and combustion gas. The cooling bath 53 on the other hand is retained by cooling water to have a temperature of 60 degrees C. or less. The reference numeral 58 denotes a supply line for the cooling water. The reference numeral 59 denotes a discharge line.

Synthetic gas as a raw material of methanol is supplied from a gasification reaction apparatus (not shown) via the synthesis raw material gas supply line A. Then, the synthetic gas is heated by the temperature adjustment means 7 in the constant temperature room 8 to have a predetermined temperature. Then, the synthetic gas is sent to the reactors 2 where methanol gas is synthesized by an action by catalysts.

Methanol gas and unreacted synthetic gas are sent from the reaction gas derivation pipe 5 to the cooler 3 and only methanol gas is extracted by liquefaction and is collected through the liquid fuel collection pipe 6. On the other hand, unreacted synthetic gas is sent via the synthetic gas supply pipe 4 to the next stage of the temperature adjustment means 7 and is further sent to the reactors 2. The reference numeral 60 denotes an outlet line of unreacted synthetic gas.

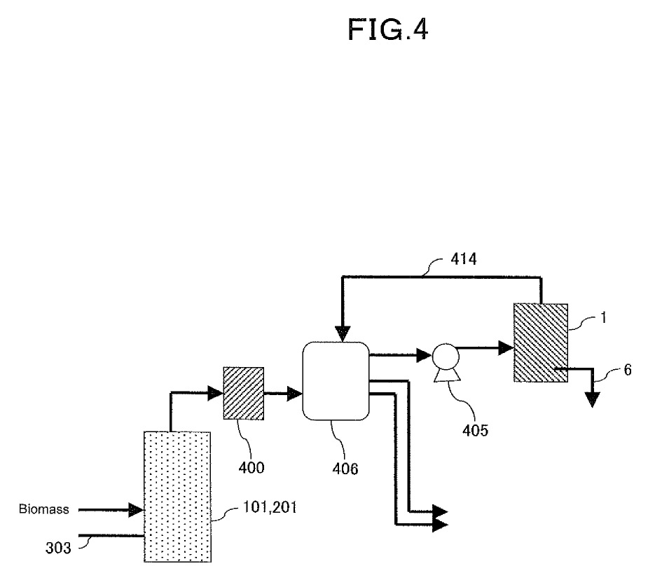

FIG. 4 schematically illustrates a process flow according to the second embodiment for carrying out the present invention. In FIG. 4, solid biomass is firstly gasified to have a gaseous state. Various biomasses can be used including, for example, industrial and agricultural wastes such as woods, construction waste, bark, paddy straw, or bagasse and kitchen waste frequently including cellulose as a main component. However, the invention is not particularly limited to them. Gasification agent generally may be water vapor, air, oxygen or the like.

The reference numeral 205 denotes a supply line for biomass as a raw material. The reference numeral 303 denotes a gasification agent supply line. Gasification agent may be water vapor and carbon dioxide for example as described later. Carbon dioxide also functions as auxiliary agent supporting the decomposition and gasification of biomass. The reference numerals 101 and 201 denote a high-temperature hot gas generation apparatus and a gasification reaction apparatus, respectively. However, other known biomass gasification methods also may be used such as the one using a fixed bed and the one using a fluid bed.

Through gasification, not only hydrogen and carbon monoxide as raw material for methanol synthesis but also valuable hydrocarbon such as methane, ethylene, or ethane and carbon dioxide are generated. In addition to these substances, minor components are also generated that are a sulfur compound such as hydrogen sulfide or carbonyl sulfide and a nitrogen compound such as ammonia or hydrogen cyanide. A sulfur compound in particular shows toxicity to a methanol synthesis catalyst and thus is optionally removed by known absorbing liquid or adsorption agent. The reference numeral 400 denotes a cleanup apparatus for this purpose.

The gas thus obtained is once stored in a gas tank 406 and is to be used as a raw material for producing methanol. Once stored, the synthetic gas is pressurized at a pressure of 0.5 to 5 MPa by a pressurization pump 405 and is guided to the liquid fuel synthesis apparatus 1. The reference numeral 6 denotes a liquid hydrocarbon collection pipe. The reference numeral 414 denotes a circulation line for unreacted gas to a gas tank. This gas includes unreacted hydrogen, carbon monoxide as well as hydrocarbon such as methane or ethylene and carbon dioxide and thus can be used as fuel for a gas engine. When the only purpose is to produce methanol, the gas may be further subjected to processes generally used in the chemical industry such as a steam reforming for conversion to hydrogen and carbon monoxide and the use of a shift reactor for adjusting a ratio between hydrogen and carbon monoxide deviating from a range suitable for methanol synthesis.

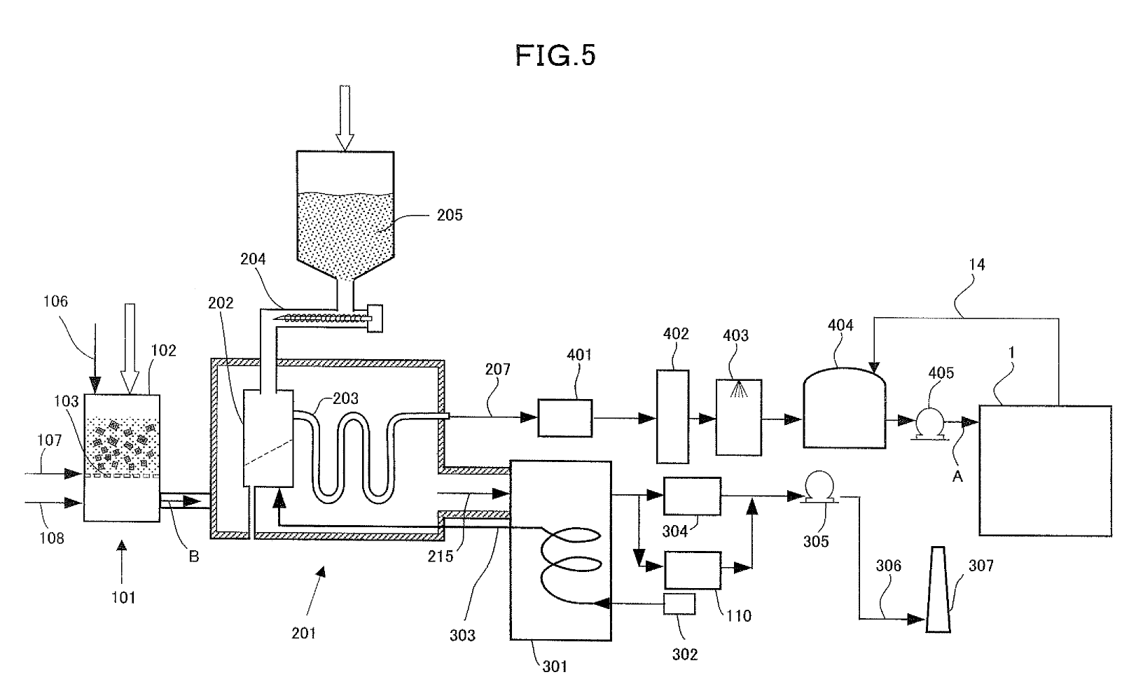

Next, FIG. 5 illustrates a configuration example of the entire liquid fuel production apparatus according to the third embodiment for carrying out the present invention. Specifically, the liquid fuel production apparatus is composed of: a biomass supply hopper 205; a gasification reaction apparatus 201; and the liquid fuel synthesis apparatus 1. The gasification reaction apparatus 201 includes therein the secondary gasification reaction pipe 203 and the primary gasification reaction room 202 having a gasification agent supply line 303 linked to the secondary gasification reaction pipe 203. The liquid fuel synthesis apparatus 1 is structured, as described above, so that the reactors 2 (not shown in FIG. 5) and the coolers 3 (not shown in FIG. 5) are arranged in a serial manner. The reactor 2 and the cooler 3 are connected to each other via the synthetic gas supply pipe 4 (not shown in FIG. 5) and the reaction gas derivation pipe 5 (not shown in FIG. 5). The cooler 3 (not shown in FIG. 5) includes the liquid fuel collection pipe 6 (not shown in FIG. 5).

The biomass supply hopper 205 is connected to the gasification reaction apparatus 201 by being connected to the primary gasification reaction room 202 via a biomass supply line 204. The gasification reaction apparatus 201 is connected to the liquid fuel synthesis apparatus 1 via the synthesis raw material gas supply line A. The biomass supply line 204 is composed of a transfer means such as a belt conveyor or a screw feeder.

In this apparatus, clean high-temperature combustion gas 109 having a temperature exceeding 900 degrees C. that is generated from biomass-ground fuel by a high-temperature hot gas generation apparatus 101 is sent to the gasification reaction apparatus 201. Then, the primary gasification reaction room 202 in the gasification reaction apparatus 201 and the secondary gasification reaction pipe 203 connected thereto are heated by heating the outer wall face.

In this example, the primary gasification reaction room 202 receives the overheat water vapor from the bottom section, wherein the overheat water vapor is generated by a waste heat boiler 301 out of emission gas 215 and also receives coarsely-ground biomass from the upper section, through the biomass supply line 204. In the primary gasification reaction room 202, the coarsely-ground biomass and overheat water vapor functioning as a gasification agent absorb, as chemical reaction heat, radiation heat from the wall of the gasification reaction room to thereby provide gasification including a overheat water vapor reforming reaction without using a catalyst.

The synthetic gas generated in the primary gasification reaction room 202 further promotes the gasification reaction of tar and soot included therein. Thus, the synthetic gas is sent to the secondary gasification reaction pipe 203 and is subsequently sent as synthetic gas to a fuel gas tank 404 and is temporarily stored. The secondary gasification reaction pipe 203 and the fuel gas tank 404 have therebetween a heat exchanger 401 for collecting heat waste, cyclone 402 for removing ash and soot, a water spray scrubber 403 for removing residual water vapor, and a pressurization pump 405.

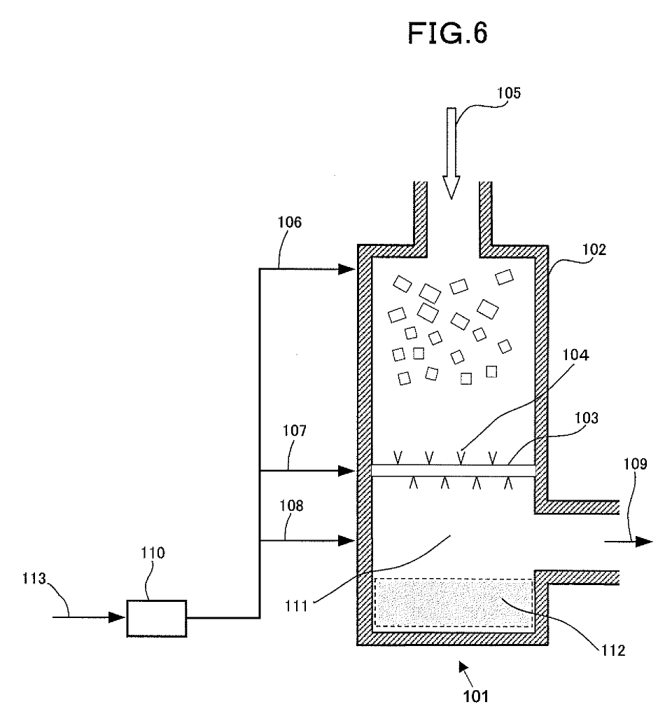

FIG. 6 is a schematic view illustrating the high-temperature hot gas generation apparatus 101. A combustion furnace 102 is structured to have a shaft furnace-type fixed floor and the lower part of the combustion furnace has a fire grate 103. Biomass as a fuel is obtained by forming chips to have an appropriate shape of about 10 cm to drop the chips through the top of the combustion furnace 102.

As combustion air, primary air 106 from the upper part, secondary air 107 from the neighborhood of the fire grate 103, and tertiary air 108 from the lower part of the fire grate 103 are supplied. The air or combustion gas in the combustion furnace 102 is caused to flow in the lower direction because the discharged high-temperature combustion gas 109 is induced by an induced draft fan at the downstream part to a chimney pipe. By this method, the temperature of the combustion in the combustion furnace reaches the highest combustion temperature in the vicinity of the upper part of the fire grate 103. The combustion gas including some combustible gas is completely combusted by the tertiary air in a furnace bottom combustion room 111 under the fire grate in a clean manner.

The cleanliness levels of the gas property of the generated high-temperature combustion gas 109 are shown in the following example.

### Oxygen (O₂) 4.2%

Carbon dioxide (CO₂) 14.6%
Hydrogen chloride (HCl) 430 ppm
Hydrogen sulfide (H₂S) 120 ppm

### Hydrocarbon (CmHn) 0%

Carbon monoxide (CO) 0%
Dioxin lower than detection limit
Combustion temperature 1220 degrees C.

The high-temperature hot gas generation apparatus 101 has an air preheater 110 that can heat the primary air 106, the secondary air 107, and the tertiary air 108 by 450 degrees C. at the maximum by the high-temperature combustion gas 109. Accordingly the high-temperature hot gas generation apparatus 101 has a function to cause even biomass chips including 60% of water to be easily combusted. Further The combustion temperature in the fire grate 103 exceeds 1300 degrees C. and thus a structure for resisting such a high temperature is provided so that the interior of the metal structure of the fire grate 103 is cooled by a part or the entirety of the secondary air 107 and then the secondary air 107 is discharged as a combustion air through many secondary air discharge orifices 104.

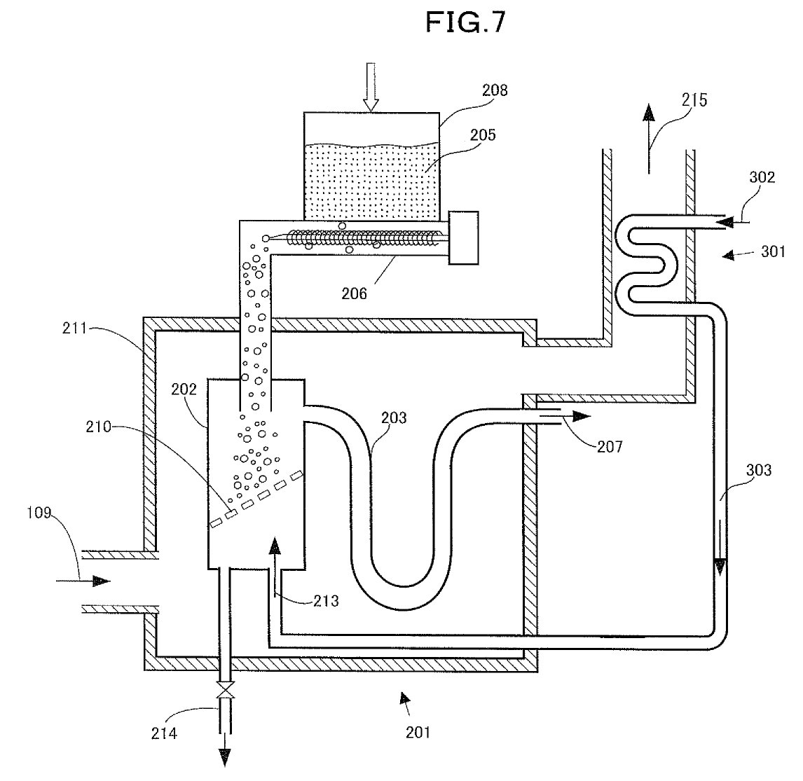

FIG. 7 is a schematic view illustrating the gasification reaction apparatus 201. The inner face of the outer wall of the gasification reaction apparatus 201 is covered by heat insulation material 211. The gasification reaction apparatus 201 includes therein the primary gasification reaction room 202 and the secondary gasification reaction pipe 203 connected thereto. Through the upper part of primary gasification reaction room 202, the coarsely-ground biomass is supplied by dropping from the biomass supply hopper 205 via the biomass supply line 204 (screw feeder in this example). On the other hand, from the bottom section of the primary gasification reaction room 202, the overheat water vapor obtained by the waste heat boiler 301 is supplied as biomass gasification agent 213. The high-temperature combustion gas 109 is introduced via the high-temperature combustion gas supply line B to the interior of the gasification reaction apparatus 201 to heat the primary gasification reaction room 202 and the secondary gasification reaction pipe 203.

In the primary gasification reaction room 202, chemical reaction between the coarsely-ground biomass and the gasification agent 213 is caused by the radiation heat from the wall of the reaction room, thereby generating the synthetic gas 207 such as H₂, CO, CH₄, C₂H₄, and CO₂.

At the middle of the lower part in the primary gasification reaction room 202, a porous plate 210 is provided that is made of a ceramic porous member or a punching copper plate for example. The coarsely-ground powder biomass about 3 mm or more remains on the porous plate 210 and is gasified for a long time. The generated synthetic gas gasified in the primary gasification reaction room 202 may include some soot and tar.

Thus, the generated gas is sent to the secondary gasification reaction pipe 203 and the remaining soot and tar are redecomposed and gasified by the gasification agent to obtain clean synthetic gas that is used as fuel gas.

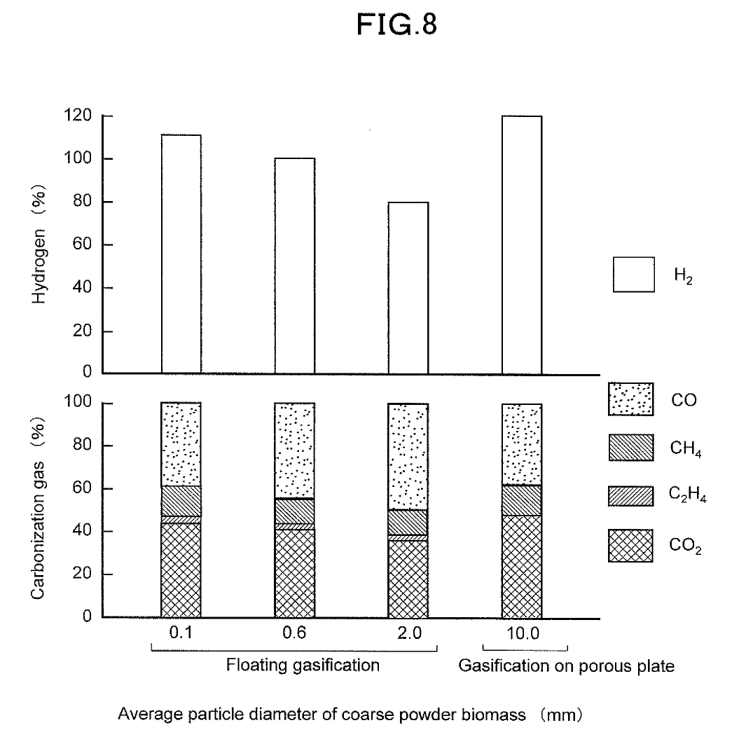

FIG. 8 illustrates a comparison of the composition between the synthetic gas by the floating gasification of the microparticle biomass and the synthetic gas by the gasification of the coarse powder biomass on the porous plate 210 in the biomass gasification apparatus of the third embodiment for carrying out the present invention. The graph shown at the lower side assumes carbonization gas composition of the synthetic gas as 100%. Since ethylene C₂H₄ means two carbon atomic molecules, the composition % was doubled to assume the total carbon gas as 100%.

The graph shown at the upper side illustrates a percentage of the generated hydrogen (H₂) to the carbonization gas 100%. Any of the results were obtained by the gasification reaction room at 900 degrees C.

The amount of hydrogen (H₂) in the upper graph depends not only by the hydrogen in the biomass (e.g., C_{1.3}H₂O_{0.9}) but also by the reaction between the hydrocarbon gas of the biomass gasified component and water vapor. For example, this can be represented by: C₂H₄+4H₂O→2CO₂+6H₂.

Thus, it can be assumed that the gasification reaction is promoted as the hydrogen amount in the upper graph increases. As can be seen from FIG. 8, in the gasification reaction in which biomass particles of 0.1 mm, 0.6 mm, and 2.0 mm float in the gasification agent, the biomass of smaller particles can be gasified more easily while the biomass of larger particles is suppressed from the promotion of the gasification reaction. However, as can be seen from FIG. 8, even the coarse powder biomass having a size of 10 mm can be subjected to the gasification reaction equal to or higher than the floating gasification by causing the biomass to remain on the porous plate to gasify the biomass. In this case, however, the biomass remaining on the porous plate requires a few minutes of gasification reaction time while microparticles require the floating gasification reaction of substantially 1 a second or less.

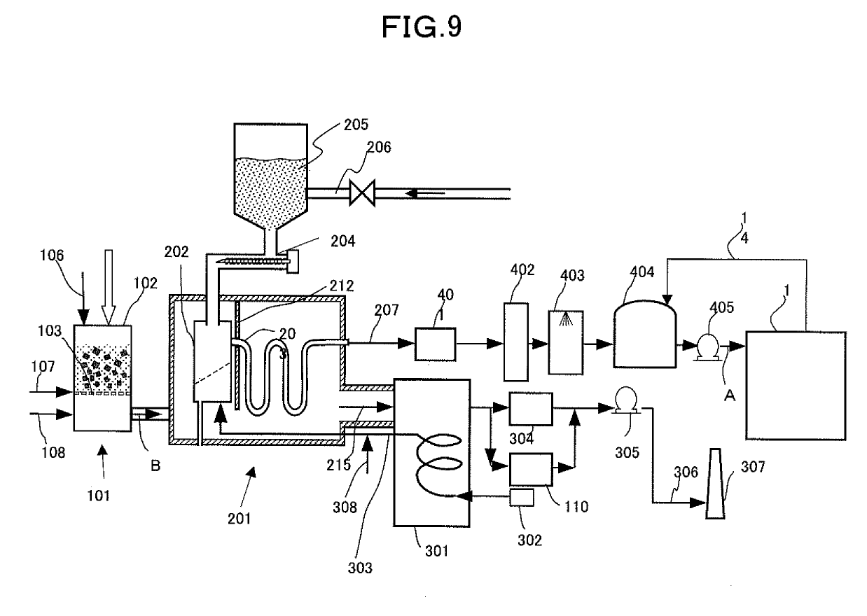

FIG. 9 shows an embodiment obtained by modifying the embodiment of FIG. 5. In order to prevent the water vapor entering from the primary gasification reaction room 202 from condensing dew to cause an unnecessary load on the gasification reaction, the biomass supply hopper 205 is linked to a coarse powder-accompanying gas supply line 206. The coarse powder-accompanying gas may be selected from among nitrogen gas, carbon dioxide, air, or the mixture thereof. When the biomass is accompanied by the coarse powder-accompanying gas that has about 2 to 15% weight ratio to the biomass in the case of carbon dioxide, about 2 to 10% weight ratio in the case of nitrogen, and about 2 to 5% weight ratio in the case of air, this can prevent the water vapor supplied to the primary gasification reaction room 202 from entering the biomass supply hopper 205, thereby preventing the humidification of the biomass in the biomass supply hopper 205. This can consequently reduce the loss of thermal energy in the gasification reaction apparatus 201, thus improving the gasification efficiency.

The coarse powder-accompanying gas may be selected in consideration of the application of generated gas. For example, carbon dioxide is advantageously used when the generated gas is used for methanol synthesis. An appropriate amount of nitrogen mixed in the accompanying gas has an effect of reducing water vapor. In considering for the calorific value required to heat water enthalpy to an overheat water vapor enthalpy of a reaction temperature, nitrogen gas is preferably used for a gas engine fuel application where a higher calorific value is prioritized. Another option is air for reasons of the availability of nitrogen gas and cost.

In this embodiment, the external space of the primary gasification reaction room 202 and the external space of the secondary gasification reaction pipe 203 are divided by a heat-resistant partition wall 212 so that the high-temperature combustion gas 109 can communicate therethrough and the heat radiation between the primary gasification reaction room 202 and the secondary gasification reaction pipe 203 can be blocked.

By the partition wall 212, the high-temperature combustion gas 109 can be supplied, in a prioritized manner, to the primary gasification reaction room 202 requiring a higher temperature.

In addition to water vapor, gasification substitute gas (carbon dioxide) 308 is supplied as a gasification agent from the gasification agent supply line 303. Specifically, the gasification agent obtained by mixing the overheat water vapor obtained by the waste heat boiler 301 with the gasification agent substitute gas 308 consisting of carbon dioxide is supplied via the gasification agent supply line 303 to the bottom section of the primary gasification reaction room 202. Carbon dioxide has some function as gasification agent at a high temperature. Carbon dioxide however causes an increase in the carbon dioxide component in the synthetic gas. When the synthetic gas is used for the synthesis of methanol, carbon dioxide in the synthetic gas is bonded to hydrogen to generate methanol. This provides, when hydrogen can be supplied from water electrolysis by natural energy for example in particular, an effect of significantly increasing the production amount of methanol synthesis by the supply of carbon dioxide.

### INDUSTRIAL APPLICABILITY

The present invention provides a new way to the use of biomass that is assumed as the most promising energy from the quantitative viewpoint among the renewable energy as a base for the sustainable society expected in the future. This technique may be widely used not only in Japan but also in foreign countries. Also according to the present invention, wood and grass having no competitive relation with food can be gasified to thereby produce methanol in an industrial manner. The resultant methanol can be used as biomass-derived methanol fuel, can be further converted to bio diesel fuel, or also can be converted to other chemical raw materials and thus has an extremely-wide industrial applicability.

What is claimed is:

1. A process for production of liquid fuel from synthetic gas that is obtained by gasification of biomass and that includes hydrogen and carbon monoxide, comprising:
a first step of causing the synthetic gas to have a contact with catalyst while being pressurized at a pressure of 0.5 to 5 MPa to synthesize liquid fuel gas;
a second step of liquefying the liquid fuel gas to collect the gas to separate the gas from unreacted synthetic gas;
a third step of again causing the unreacted synthetic gas to have a contact with catalyst while being pressurized at a pressure of 0.5 to 5 MPa to synthesize liquid fuel gas to subsequently liquefy the resultant liquid fuel gas to collect the gas to separate the gas from unreacted synthetic gas; and
a fourth step of repeating the third step.

2. The process for production of liquid fuel according to claim 1,
wherein the liquid fuel is methanol.

3. The process for production of liquid fuel according to claim 1,
wherein the liquid fuel is liquid hydrocarbon.

4. A liquid fuel synthesis apparatus, comprising:
a plurality of reactors and a plurality of coolers that are arranged in a serial manner,
wherein the reactors and the coolers are connected to one another via a synthetic gas supply pipe and a reaction gas derivation pipe, and
the coolers include liquid fuel collection pipes.

5. The liquid fuel synthesis apparatus according to claim 4, wherein an inlet-side of the synthetic gas supply pipe of the reactor has a temperature adjustment means.

6. The liquid fuel synthesis apparatus according to claim 4, wherein the reactors are parallely arranged in a constant temperature room in which a temperature can be adjusted.

7. The liquid fuel synthesis apparatus according to claim 4, wherein the reactor maintains the temperature of the interior at an appropriate temperature by subjecting reaction heat generated by a synthetic reaction of the synthetic gas to a heat exchange with surrounding atmosphere.

8. The liquid fuel synthesis apparatus according to claim 4, wherein a constant temperature room includes parallely-arranged reactors for maintaining the inner temperature at an appropriate temperature by subjecting reaction heat generated by the synthetic reaction to a heat exchange with surrounding atmosphere.

9. The liquid fuel synthesis apparatus according to claim 7 or 8,
wherein an inlet of the synthetic gas supply pipe has a temperature adjustment means and a constant temperature room includes the reactors for maintaining the inner temperature at an appropriate temperature by subjecting reaction heat generated by the synthetic reaction to a heat exchange with surrounding atmosphere, the reactors being parallely-arranged.

10. The liquid fuel synthesis apparatus according to claim 4, wherein the coolers subject gas or liquid having a temperature equal to or lower than a predetermined temperature to a heat exchange.

11. The liquid fuel synthesis apparatus according to claim 4, wherein the coolers are stored in a cooling bath filled with cooling water having a predetermined temperature or less.

12. The liquid fuel synthesis apparatus according to any of claims 4 to 11,
wherein the liquid fuel is methanol.

13. The liquid fuel synthesis apparatus according to any of claims 4 to 11,
wherein the liquid fuel is liquid hydrocarbon.

14. A liquid fuel production apparatus, comprising:
a biomass supply hopper;
a gasification reaction apparatus; and
a liquid fuel synthesis apparatus,
wherein the gasification reaction apparatus includes therein a secondary gasification reaction pipe and a primary gasification reaction room that has a gasification agent supply line linked to the secondary gasification reaction pipe,
the liquid fuel synthesis apparatus is composed of a plurality of reactors and a plurality of coolers that are arranged in a serial manner, the reactors and the coolers are connected to one another via a synthetic gas supply pipe and a reaction gas derivation pipe, and the coolers include liquid fuel collection pipes,
the biomass supply hopper is connected to the gasification reaction apparatus by being connected to the primary gasification reaction room via a biomass supply line, and
the gasification reaction apparatus is connected to the liquid fuel synthesis apparatus via a synthesis raw material gas supply line.

15. The liquid fuel production apparatus according to claim 14,
wherein the biomass supply hopper includes a coarse powder-accompanying gas supply line.
